(19) United States
(12) Patent Application Publication
(10) Pub. No.: US 2020/0033361 A1
(43) Pub. Date: Jan. 30, 2020

(54) Detection and Quantitation Method for Proteomics of Post-Translational Modifications

(71) Applicant: Sichuan University, Chengdu (CN)

(72) Inventor: Lunzhi DAI, Chengdu (CN)

(73) Assignee: Sichuan University, Chengdu (CN)

(21) Appl. No.: 16/575,588

(22) Filed: Sep. 19, 2019

Related U.S. Application Data
(63) Continuation-in-part of application No. PCT/CN2017/081059, filed on Apr. 19, 2017.

(30) Foreign Application Priority Data
Mar. 24, 2017 (CN) 201710183629.8

(51) Int. Cl. G01N 33/68; G01N 1/28

(52) U.S. Cl. CPC G01N 33/6848; G01N 1/28; G01N 2800/00; G01N 2560/00; G01N 2440/12

(57) ABSTRACT

The present disclosure relates to the technical field of comparative proteomics, in particular to a detection and quantitation method for proteomics of post-translational modifications. With this method, the protein samples to be studied and internal standards are labeled with isobaric tandem mass tags, and tandem mass spectrometry analysis is carried out for the labeled peptide mixture, wherein the internal standard is a peptide mixture rich in post-translational modifications to be detected. Through this method, the signal of peptides containing the post-translational modifications to be detected can be amplified under the situation that mass spectrometer sensitivity is unchanged, and enrichment of the post-translational modification peptides is not needed. The probability of detecting the peptides containing the post-translational modifications to be detected by mass spectrometer and being selected for subsequent MS/MS analysis is increased.

### CROSS-REFERENCE TO RELATED APPLICATIONS

The present disclosure is a continuation-in-part application based on international patent application No. PCT/CN2017/081059, filed on Apr. 19, 2017 and entitled “Detection and Quantitation Method for Proteomics of Post-translational Modifications”, and the international patent application claims the priority to the Chinese patent application with the application number CN201710183629.8, filed on Mar. 24, 2017 with Chinese Patent Office, and entitled “Detection and Quantitation Method for Proteomics of Post-translational Modifications”, the contents of which are incorporated by reference herein in entirety.

### TECHNICAL FIELD

The present disclosure relates to the technical field of comparative proteomics, in particular to a detection and quantitation method for proteomics of post-translational modifications.

### BACKGROUND ART

Protein post-translational modification is an important mechanism for regulating genetic expression and protein function, and play important roles in numerous biological processes and pathways. Abnormal regulation of protein post-translational modifications is closely related to occurrence and development of many diseases. In past decades, many new post-translational modification types of proteins were characterized, providing an important foundation for high-throughput proteomic analysis of protein post-translational modifications, and understanding novel functions of protein post-translational modifications.

As most protein post-translational modifications have extremely low abundance, it brings about huge difficulty to qualitative and quantitative analysis of post-translational modifications in a high-throughput manner. At present, mass spectrometry-based proteomic technology is an important tool for studying post-translational modifications. Specifically, peptides containing specific protein post-translational modification are first enriched using a specific antibody, then the enriched peptides are subjected to mass spectrometry analysis. However, there are still some shortcomings and defects in the current strategy for analyzing post-translationally modified proteomes: (1) a large amount of biological samples is required. For the current strategy, at least mg level of protein extracts are needed for studying one type of post-translational modification, while many trace biological samples cannot meet this requirement (2) A large amount of specific antibodies or other enrichment reagents, such as TiO₂, are consumed to enrich the post-translationally modified peptides. The antibodies and the enrichment reagents are quite expensive, and it is also quite difficult to acquire a specific enrichment reagent, increasing the difficulty in the proteomic analysis of post-translational modifications. (3) The qualitative and quantitative detection has a relative low throughput. As the peptides where the post-translational modifications are located will affect the binding of pan antibodies, many post-translationally modified peptides cannot be enriched, resulting in a quite low detection throughput by mass spectrometer. (4) Repeatability and reproducibility are poor. The enrichment of modified peptides is quite difficult to operate, and different technological levels of operators cause quite big offsets to experiment results. Besides, the acquisition process of mass spectrometry will also cause certain offset to results. (5) Only one type of post-translational modifications can be analyzed in one time using the current strategy, and if a plurality of types of post-translational modifications of a single sample are to be studied, dosage of the sample, dosage of the antibody, and the analyzing time will be multiplied, finally greatly increasing the cost of the whole research. (6) The quantitation methods for protein post-translational modifications are limited, mainly including SILAC labeling quantitation, isobaric labeling quantitation (for example, TMT, iTRAQ) and label-free quantitation. These methods are not universal and can only be used in some situations. For examples, The SILAC labeling quantitation is usually only suitable to the proteomics of post-translational modifications of culture cells; the isobaric labeling quantitation sometimes is not suitable to study modified proteome, because it is quite costly to perform isobaric labeling on a large amount of biological samples, and after labeling, the efficiency of enriching the modified peptides is greatly influenced; the label-free quantitation is a strategy currently having a better prospect, but it has a relative low degree of accuracy, and poor reproducibility. As there are many shortcomings and defects above in the current methods, it urgently needs a new strategy for quantitative proteomics of post-translational modifications.

### SUMMARY

An object of the present disclosure is, for example, to provide a detection and quantitation method for proteomics of post-translational modifications, so as to solve the above problems.

A detection and quantitation method for proteomics of post-translational modifications includes labeling protein samples to be detected and an internal standard with isobaric tandem mass tags, and carrying out tandem mass spectrometry analysis for a labeled peptide mixture, In the above, the internal standard is a peptide mixture rich in post-translational modifications to be detected.

The present disclosure further provides a method for diagnosing diseases caused by dysregulation of post-translational modifications, including detection and/or quantitation for proteomics of post-translational modifications in a subject sample using the method disclosed in the present disclosure.

The detection and quantitation method for proteomics of post-translational modifications provided in the present disclosure does not need enrichment technologies using, for example, antibodies, immobilized metal affinity chromatography or TiO₂, significantly reducing experimental costs, and shortening an experimental procedure. The isobaric labelling creates different isobaric tandem mass tags on peptides from internal standard and samples to be studied, respectively, to identify the resource of samples and quantify the proteins and peptides between samples. When mixed with internal standard, the identical modified peptides, from either samples to be studied or internal standard, would appear as single composite peaks in the MS1 spectra and provide stronger signal to be selected for MS/MS analysis.

### BRIEF DESCRIPTION OF DRAWINGS

In order to more clearly illustrate technical solutions in embodiments of the present disclosure or the prior art, accompanying drawings which need to be used for description of the embodiments of the present disclosure or the prior art will be introduced briefly below Apparently, the accompanying drawings in the description below merely show one or more embodiments of the present disclosure. A person ordinarily skilled in the art still could obtain other relevant drawings in light of these accompanying drawings, without using inventive effort.

FIG. 1 is a flowchart of a detection and quantitation method for proteomics of post-translational modifications provided in the present disclosure. This flowchart is suitable to a method of performing chemical modification labeling at a protein level. Samples are mainly divided into two groups, that is, a sample group to be studied and a group of internal standard.

FIG. 2 is a flowchart of a detection and quantitation method for proteomics of post-translational modifications provided in the present disclosure, this flowchart is suitable to a method of performing chemical modification labeling at a peptide level. Samples are mainly divided into two groups, that is, a sample group to be studied and a group of internal standard.

FIG. 3 is a MS/MS mass spectrum corresponding to an acetylated peptide in comparative acetylomics and 2-hydroxyisobutyrylomics in livers of mice on a high fat diet in Example 1.

FIG. 4 shows regions where reporter groups are located in the MS/MS mass spectrum corresponding to an acetylated peptide in comparative acetylomics and 2-hydroxyisobutyrylomics in livers of mice on a high fat diet in Example 1.

FIG. 5 shows a MS/MS mass spectrum corresponding to a dimethylated peptide in comparative dimethylomics in livers of mice on a high fat diet in Example 2.

FIG. 6 shows regions where reporter groups are located in the MS/MS mass spectrum corresponding to a dimethylated peptide in comparative dimethylomics in livers of mice on a high fat diet in Example 2.

FIG. 7 shows a MS/MS mass spectrum corresponding to a phosphorylated peptide in quantitative phosphoproteomics in colorectal cancer sample in Example 3.

FIG. 8 shows regions where reporter groups are located in the MS/MS mass spectrum corresponding to a phosphorylated peptide in quantitative phosphoproteomics in colorectal cancer sample in Example 3.

FIG. 9 shows a MS/MS mass spectrum corresponding to an acetylated peptide in quantitative acetylomics in colorectal cancer sample in Example 4.

FIG. 10 shows regions where the complementary TMTc reporter groups are located in the MS/MS mass spectrum corresponding to an acetylated peptide in quantitative acetylomics in colorectal cancer sample in Example 4.

FIG. 11 shows regions where reporter groups are located in the MS/MS mass spectrum low mass region corresponding to an acetylated peptide in quantitative acetylomics in colorectal cancer sample in Example 4.

### DETAILED DESCRIPTION OF EMBODIMENTS

In order to overcome shortcomings and defects of conventional proteomic approaches for studying post-translational modifications, such as using a large amount of biological samples, pan and specific antibodies, and only detecting one type of modification in one time, high overall costs, long time, difficult operation, and poor repeatability, the present disclosure provides a qualitative and quantitative proteomic method based on amplification of a post-translationally modified peptide signal through assistance of the internal standard. In the present disclosure, an internal standard is a doubly chemically labeled sample used to amplify the signal of post-translationally modified peptide, such that it reaches a range detectable by bio-mass spectrometry.

The present disclosure relates to a detection and quantitation method for proteomics of post-translational modifications, and its use in qualitative and quantitative analysis of post-translational modifications. The method includes:
labeling protein samples to be studied and an internal standard with isobaric tandem mass tags, and carrying out tandem mass spectrometry analysis for a labeled mixture;
in the above, the internal standard is a peptide mixture rich in post-translational modifications to be detected.

In one or more embodiments, the method of the present disclosure includes performing enzymolysis on the protein samples to be studied in advance to generate peptides, labeling the generated peptides and the internal standard respectively with the isobaric tags, mixing the labeled peptides before performing mass spectrometry analysis, and carrying out qualitative and quantitative analysis on the post-translational modifications using TMTc reporters in the high mass range and reporter ions in the low mass range.

In one or more embodiments, the internal standard is a peptide mixture rich in the post-translational modifications to be detected.

In one or more embodiments of the present disclosure, the post-translational modifications includes following types: acylation (for example, acetylation, formylation, and/or palmitoylation of lysine), alkylation (for example, lysine methylation, arginine methylation, cysteine prenylation), phosphorylation, ubiquitylation, glycosylation, sulfation, selenylation, S-nitrosylation, adenylation (for example, AMPylation, UMPylation), hydroxylation (for example, lysine hydroxylation, proline hydroxylation), citrullination, carbamylation, amidation (for example, glutamic acid glycinylation, methylamidation) and iodization (for example, tyrosine iodization).

In one or more embodiments of the present disclosure, the post-translational modifications to be detected includes at least one from the following: acylation, alkylation, phosphorylation, ubiquitylation, glycosylation, sulfation, selenylation, S-nitrosylation, adenylation, hydroxylation, iodization, citrullination and carbamylation, amidation and iodization.

In one or more embodiments, the isobaric tandem mass tags include at least one from TMT (tandem mass tags), iTRAQ (isobaric tags for relative and absolute quantification), DiART (deuterium isobaric amine reactive tags), CIT (caltech isobaric tags), CILAT (cleavable isobaric labeled affinity tags), DiLeu (N,N-dimethyl leucines) quantitation tags, IPTL (isobaric peptide termini labelling) tags, QITL (quantitation by isobaric terminal labeling) tags, IVTAL (in vivo terminal amino acid labeling), and EASI-TAG (Easily Abstractable Sulfoxide-based Isobaric tag). Any tag that can realize the isobaric tandem mass labeling can be used in the technical solution of the present disclosure.

As used in the present disclosure, the term “isobaric labeling” is a mass spectrometry strategy used in quantitative proteomics, which involves labeling peptides or proteins with various chemical groups of identical mass (isobaric). The various chemical groups are different in distribution of heavy isotopes around structures thereof. These tags, commonly referred to as tandem mass tags, are designed so that the mass tag is cleaved at a specific linker region upon high-energy CID (HCD) during tandem mass spectrometry yielding reporter ions of different masses.

In one or more embodiments, the reporter ions are reporter ions in a low mass area and the complementary reporter ions containing peptide sequences (TMTc) in a high mass area.

In one or more embodiments, 3 channels of isobaric tags (126, 129 and 131) are preferred to minimize and eliminate the influences of co-isolated precursors and isotope impurities. The signals of TMTc are used for the characterization of modified peptides, and the reporter ions or TMTc are used for peptide quantification.

The post-translational modifications to be detected on the internal standard may be obtained through artificial chemical labeling of proteins or peptides. The internal standard also may be proteins or peptides containing the post-translational modifications to be detected obtained upon separation through other existing technologies (for example, performing enrichment using antibodies). The internal standard also may be chemically synthesized peptides containing the post-translational modifications to be detected.

In the present disclosure, the probability that the modified peptides are detectable by mass spectrometer and further subjected to MS/MS analysis is improved by adding the internal standard to the samples to be studied. In the above, in the peptide mixture entering the mass spectrometry analysis, the effective components are from both singly chemically labeled group and doubly chemically labeled group.

The singly chemically labeled group is obtained by labeling the protein samples to be studied with the isobaric tandem mass tags.

The doubly chemically labeled group is the internal standard and obtained by performing sequential chemical modification labeling (first chemical labeling) and isobaric labeling (second chemical labeling).

In one or more embodiments of the present disclosure, there may be any number of internal standard and samples to be studied, but the number should not be more than the labelable number of the isobaric tags. For example, if a 10-tag TMT reagent is chosen as the isobaric tags, the total number of the internal standard and the samples to be studied at most is no more than 10.

When the tandem mass spectrometry analysis is performed, MS1 signal is a composite signal of the same peptide of the internal standard and the samples to be studied. Due to the first chemical modification labeling, the abundance of the modified peptides in the internal standard is greatly improved. After it is mixed with the modified peptides in the samples to be studied, the MS1 signals are stacked, and the endogenous modified peptide signals can be amplified in the situation that the sensitivity of mass spectrometer is unchanged, and the enrichment of the post-translational modification is not performed. As a result, the probability of being detectable by mass spectrometer and further subjected to MS/MS analysis is improved.

At the MS/MS level, the peptide signals of the internal standard and the samples to be studied are distinguished by reporter ions, wherein if there is a signal of the reporter ion, it indicates that this peptide exists in the corresponding sample, and if there is no signal of the reporter ion, it indicates that this peptide does not exist in the corresponding sample. The ratio of reporter ions reflects a relative abundance of the peptide in different samples. In most instances, to reduce to false positive results, the signal of TMTc is used for peptide characterization, and the reporter ions in the low mass region are used for peptide quantification. Besides, the present disclosure further has the characteristics such as simultaneous qualitative and quantitative analysis of many types of post-translational modifications using trace samples.

Through the above analysis of the experimental principle, it is easy to conceive of that the internal standard is a single type of protein or a mixture of many types of proteins. Theoretically, it is advantageous that the proteins used in the internal standard are as similar or close to the proteins in the samples to be studied as possible. For example, the internal standard and the samples to be studied are extracted proteins of a single source, or the internal standard is a mixture of the samples to be studied and other samples.

Regarding many shortcomings and defects of existing qualitative and quantitative analysis method for protein post-translational modifications, the present disclosure has many revolutionary breakthroughs: first, as the internal standard increases the MS1 signals of modified peptides, the starting protein material in the present disclosure can be as low as microgram level; second, the qualitative and quantitative analysis of post-translational modification can be achieved in the present disclosure without enrichment with specific antibodies or other reagents, and quantitation of proteins and protein post-translational modification is completed simultaneously in a single experiment, greatly reducing costs and mass spectrometry analysis time; third, in the present disclosure, simultaneous qualitative and quantitative analysis of many types of protein post-translational modifications can be achieved, greatly reducing the machine-hour of mass spectrometry, and saving a lot of detection fees; fourth, the reproducibility and repeatability of experiment are good, and as the enrichment with antibodies is not needed, errors of human operations are greatly reduced; fifth, the present disclosure overcomes restrictions of use of existing methods such as SILAC method, conventional isobaric quantitation method, and label-free quantitation method, and is almost not restricted by biological sample types.

In one or more embodiments of the present disclosure, the internal standard, is a peptide mixture obtained by chemical modification labeling of the initial protein extracts and then enzymolysis, and/or a peptide mixture obtained by enzymolysis of the initial protein extracts and then chemical modification labeling, and/or a mixture of artificially synthesized peptides containing post-translational modifications to be detected, and/or a peptide mixture obtained by enriching modified peptides using antibodies or other enrichment reagents, and/or a peptide mixture obtained by enzymatic labeling of the initial protein extracts or peptides, and then enzymolysis.

In one or more embodiments of the present disclosure, the chemical modification labeling is chemical labeling, with one or more chemical modification labeling reagents, on the initial protein extracts or the peptides obtained by enzymolysis of the initial protein extracts.

For example, the chemical modification labeling reagent includes at least one of: acylation reagent, alkylation reagent, phosphorylation reagent, glycosylation reagent, ubiquitination reagent, sulfation reagent, selenylation reagent, adenylation reagent, S-nitrosylation reagent, hydroxylation reagent, carbamylation reagent, amidation reagent and iodization reagent, and enzymes that can cause the above modification. For example, the initial protein extracts are from the protein samples to be studied, and/or a mixture of recombinant proteins; for example, the initial protein extracts is a mixture obtained by dimensionality reduction of mixed protein samples to be studied; for example, technical methods used in the dimensionality reduction include HPLC, anion/cation exchange column or C18 column.

For example, the peptides obtained by enzymolysis of the initial protein extracts undergoes dimensionality reduction before chemical modification; for example, technical methods used in the dimensionality reduction include dimensionality reduction using HPLC, anion/cation exchange column or C18 column.

In one or more embodiments of the present disclosure, the acylation reagent includes: the derivatives of fatty acids and/or aromatic acids; chemical modification labeling reagents, when performing acylation labeling, should be able to react with proteins or peptides in a mild condition, to obtain specifically modified proteins or peptides at a high yield.

The derivatives include one or more from active esters, acyl halides, anhydrides, acyl coenzyme A, and high energy compounds capable of reacting with primary amino, secondary amino and hydroxyl group of a specific amino acid; For example, the specific amino acids labeled with the acylation reagent include following types: lysine, histidine, threonine, serine, tyrosine, arginine, tryptophan, 5-hydroxylysine and protein terminal amino acids.

For example, the fatty acids and/or aromatic acids include one or more from acetic acid (64-19-7), propanoic acid (79-09-4), butyric acid (107-92-6), 2-hydroxyisobutyric acid (594-61-6), malonic acid (141-82-2), succinic acid (110-15-6), glutaric acid (110-94-1), crotonic acid (107-93-7), 3-hydroxybutyric acid (300-85-6, 625-72-9, 6168-83-8), pyruvic acid (127-17-3), phosphoenolpyruvic acid (9067-77-0), oxaloacetic acid (328-42-7), citric acid (77-92-9), cis-aconitic acid (585-84-2), isocitric acid (320-77-4), malic acid (6915-15-7), fumaric acid (110-17-8), oxalosuccinic acid, lactic acid (50-21-5), 2-phosphoglyceric acid, 3-phosphoglyceric acid, diphosphoglyceric acid (138-81-8), galacturonic acid, methylmalonic acid (516-05-2), hydromethylglutaric acid (503-49-1), 2-ketobutyric acid (600-18-0), 2-hydroxybutyric acid (600-15-7), 4-pyridoxic acid (82-82-6), 3-methyl-2-oxobutyric acid (759-05-7), p-hydroxyphenylacetic acid (156-38-7), 3-hydroxylauric acid (1883-13-2), 2-methylcitric acid (6061-96-7), 3-hydroxyl-tetradecanedioic acid (73179-89-2), 2-hydroxymethylbutyric acid (4374-62-3), 3-hydroxyphenylacetic acid (621-37-4), hexanedioic acid (124-04-9), N-(3-methyl-1-oxo-2-butenyl)glycine (33008-07-0), 3,4-dihydroxyphenylpropionic acid (1078-61-1), 2-hydroxy sebacic acid (103963-71-9), 2-hydroxy-2-methylsuccinic acid (597-44-4), 3-hydroxyglutaric acid (638-18-6), homoveratric acid (93-40-3), N-(2-furoyl)glycine (5657-19-2), 2,3-dihydroxybenzoic acid (303-38-8), 2-isopropylmalic acid (3237-44-3), 2-hydroxy-3-methylbutyric acid (4026-18-0), 3-hydroxydodecanedioic acid (34574-69-1), 2-methylglutaric acid (617-62-9), 3A-hydroxy-7-oxo-5B-cholanic acid (4651-67-6), octanoic acid (124-07-2), vanillic acid (121-34-6), 7-hydroxyoctanoic acid (17173-14-7), 3-methyl-2-oxovaleric acid (1460-34-0), 2-methyl-3-hydroxybutyric acid (473-86-9), 2,4-dihydroxybutyric acid (1518-62-3), 2,3-dihydroxybutanoic acid (759-06-8), p-hydroxybenzoic acid (99-96-7), n-decanoic acid (334-48-5), chenodeoxycholic acid (474-25-9), 2-hydroxymethylpropionic acid (1910-47-0), 3,4-dihydroxybutyric acid (51267-44-8), 3-hydroxyhexanedioic acid (14292-29-6), 3-hydroxysebacic acid (73141-46-5), 3-methylglutaconic acid (5746-90-7), 5-hydroxyhexanoic acid (44843-89-2), 3-hydroxypentanoic acid (10237-77-1), N-acetylglycine (543-24-8), hexanoic acid (142-62-1), urocanic acid (104-98-3), 3β-hydroxy-A5-cholenic acid (5255-17-4), 2-hydroxy-3-methylvaleric acid (488-15-3), (2S)-2-hydroxy-hexanedioic acid (18294-85-4), 3-hydroxy-octanedioic acid (73141-47-6), 1b,3a,12a-trihydroxy-5b-cholinic acid (80434-32-8), arabinonic acid (13752-83-5), 3-methylglutarylcarnitine (102673-95-0), 3-methyladipic acid (3058-01-3), D-3-phenyllactic acid (7326-19-4), galactonic acid (13382-27-9), cinnamic acid (621-82-9, 140-10-3), phosphoenolpyruvic acid (138-08-9), L-pyroglutamic acid (98-79-3), sarcosine (107-97-1), 3-methoxy-4-hydroxymandelic acid (55-10-7), coproporphyrin III dihydrochloride (14643-66-4), elaidic acid (112-79-8), chondroitin sulfate (9007-28-7), decenedioic acid (72879-22-2), 2-hydroxyglutaric acid (2889-31-8), trihydroxybutanoic acid (13752-84-6), orotic acid (65-86-1), 3,5-dihydroxy-3-methylpentanoic acid (150-97-0), N-acetylneuraminic acid (131-48-6), quinolinic acid (89-00-9), protoporphyrin (553-12-8), furoic acid (88-14-2), cholic acid (81-25-4), glutaconic acid (1724-02-3), ethylmalonic acid (601-75-2), dodecanedioic acid (693-23-2), gluconic acid (526-95-4), D-pantothenic acid (79-83-4), orotidine 5′-monophosphate (2149-82-8), palmitic acid (57-10-3), palmitoyl carnitine (2364-67-2), 2-oxoadipic acid (3184-35-8), deoxycholic acid (83-44-3), glycodeoxycholic acid (360-65-6), citraconic acid (498-23-7), 4,6-dioxoheptanoic acid (51568-18-4), glycochenodeoxycholic acid (640-79-9), lauric acid (143-07-7), L-acetylcarnitine (3040-38-8), phenylpyruvic acid (156-06-9), oleic acid (112-80-1), α-ketoglutaric acid (328-50-7), phenylacetic acid (103-82-2), mucic acid (526-99-8), coproporphyrin 1 (531-14-6), decanoylcarnitine (1492-27-9), glucaric acid (25525-21-7), 2-hydroxy-4-methylvaleric acid (498-36-2), L-malic acid (97-67-6), L-proline (147-85-3), maleic acid (110-16-7), L-lactic acid (79-33-4), 3-indoleacetic acid (87-51-4, 1821-52-9), heptanoic acid (111-14-8), o-hydroxyphenylacetic acid (614-75-5), linoleic acid (60-33-3), N-isovalerylglycine (16284-60-9), 3B-ursodeoxycholic acid (78919-26-3), homogentisic acid (451-13-8), glycocholic acid (475-31-0), glyceric acid (473-81-4), formic acid (64-18-6), 2,5-dihydroxybenzoic acid (490-79-9), (3R)-3-(3-methylbutanoyloxy)-4-(trimethylammonio) butanoate (31023-24-2), 4-methylvaleric acid (646-07-1), S-2-hydroxypentanedioic acid (13095-48-2), 4-methyl-2-oxovaleric acid (816-66-0), glycolithocholic acid (474-74-8), glycolic acid (79-14-1), homovanillic acid (306-08-1), glyoxylic acid (298-12-4), folic acid (59-30-3), glycine (6556-12-3), 3-hydroxypropionic acid (503-66-2), hexanoyl glycine (24003-67-6), DL-mandelic acid (90-64-2), acetylcamitine (6418-78-6), 4-hydroxyphenylpyruvic acid (156-39-8), glycoursodeoxycholic acid (64480-66-6), 3-oxobutyric acid (541-50-4), L-carnitine (541-15-1), piperidine-2-carboxylic acid (535-75-1), N,N-dimethylglycine (1118-68-9), 3-hydroxy-2-methylpropanoic acid (2068-83-9), D-biotin (58-85-5), betaine (107-43-7), bilirubin (635-65-4), 4-hydroxybutyric acid (591-81-1), 2-hydroxycaprylic acid (617-73-2), hippuric acid (495-69-2), L-2-pipecolic acid (3105-95-1), isolithocholic acid (1534-35-6), glycyl-L-proline (704-15-4), L-hydroxyproline (51-35-4), 2-methyl-2-hydroxypropanoic acid (594-61-6), isobutyrylglycine (15926-18-8), S-sulfo-L-cysteine (1637-71-4), DL-3-hydroxy kynurenine (484-78-6), hyodeoxycholic acid (83-49-8), hydroxyphenylacetyl glycine (28116-23-6), isobutyryl-L-carnitine (25518-49-4), DL-homocysteine (454-29-5), L-homocarnosine (3650-73-5), L-leucic acid (13748-90-8), L-3-phenyllactic acid (20312-36-1), 3-hydroxymandelic acid (17119-15-2), 3-methylglutaric acid (626-51-7), β-hydroxyisovaleric acid (625-08-1), 3-(4-hydroxyphenyl)lactatic acid (306-23-0), L-caproyl carnitine (22671-29-0), glycyl-L-leucine (869-19-2), hyocholic acid (547-75-1), lithocholic acid (434-13-9), 5-hydroxyindole-3-acetic acid (54-16-0), hydrocinnamic acid (501-52-0), N-acetyl-L-alanine (97-69-8), nonadecanoic acid (646-30-0), DL-β-phenyllactic acid (828-01-3), propionyl glycine (21709-90-0), azelaic acid (123-99-9), orotidine (314-50-1), L-octanoylcamitine (25243-95-2), sebacic acid (111-20-6), phytanic acid (14721-66-5), D-pyroglutamic acid (4042-36-8), myristic acid (544-63-8), 3-phosphoglyceric acid (820-11-1), N-butyrylglycine (20208-73-5), N-acetyl-L-asparaginic acid (997-55-7), phenaceturic acid (500-98-1), 4-hydroxymandelic acid (1198-84-1), propionyl carnitine (17298-37-2), sialyllactose (35890-38-1), pentadecanoic acid (1002-84-2), stearic acid (57-11-4), ureidosuccinic acid (13184-27-5), 2-hydroxy hippuric acid (487-54-7), nonanoic acid (112-05-0), stearoyl carnitine (1976-27-8), L-2-aminocaproic acid (327-57-1), 3-hydroxycinnamic acid (588-30-7), DL-O-phosphoserine (17885-08-4), methylsuccinic acid (498-21-5), tetrahydrofolic acid (135-16-0), tretinoin (302-79-4), 3,4-dihydroxybenzoic acid (99-50-3), 2-hydroxyvaleric acid (617-31-2), 2-oxohexanoic acid (2492-75-3), 2-oxopentanoic acid (1821-02-9), 3,4-dihydroxymandelic acid (775-01-9), p-aminohippuric acid (61-78-9), 5-methoxysalicylic acid (2612-02-4), benzoic acid (65-85-0), isobutyric acid (79-31-2), valproic acid (99-66-1), o-acetylsalicylic acid (50-78-2), 3-chloro-L-tyrosine (7423-93-0), N-acetyl-L-cysteine (616-91-1), 3-aminobenzoic acid (99-05-8), aspartame (22839-47-0), salicylic acid (69-72-7), 6-aminocaproic acid (60-32-2), N-(iminomethyl)-L-glutamic acid (816-90-0), pimelic acid (111-16-0), monoethyl glutarate (1501-27-5), N-(3-phenylpropionyl)glycine (56613-60-6), N-acetyl-L-tyrosine (537-55-3), D-ribonic acid (642-98-8), trigonelline (535-83-1), 4,8-dihydroxyquinoline-2-carboxylic acid (59-00-7), L-valine (72-18-4), undecanedioic acid (1852-04-6), n-valeric acid (109-52-4), suberic acid (505-48-6), L-citrulline (372-75-8), sulfolithocholic acid (34669-57-3), trans-4-hydroxycyclohexyl acetate (68592-23-4), tridecanoic acid (638-53-9), succinyl adenosine (4542-23-8), uroporphyrin III (18273-06-8), ursocholic acid (2955-27-3), L-tryptophan (73-22-3), trans-2-dodecenedioic acid (6402-36-4), uroporphyrin 1(607-14-7), S-(5′-adenosine)-L-homocysteine (979-92-0), threonic acid (3909-12-4), docosanoic acid (112-85-6), ursodesoxycholic acid (128-13-2), trans-ferulic acid (537-98-4), succinyl glycine (60317-54-6), 3-hydroxy-4-methoxycinnamic acid (537-73-5), L-tartaric acid (87-69-4), trans-aconitic acid (4023-65-8), N-crotonyl glycine (35842-45-6), DL-2-aminooctanoic acid (644-90-6), L-cysteinesulfinic acid (1115-65-7), D-alanine (338-69-2), D-2-hydroxypropionic acid (10326-41-7), 6-phosphogluconic acid (921-62-0), N6,N6,N6-trimethyl-L-lysine (19253-88-4), 3,4-dihydroxyphenylacetic acid (102-32-9), 3-hydroxy-2-carbonyl propionic acid (1113-60-6), pyrrolidine hydroxycarboxylic acid (22573-88-2), 2,6-diaminopimelic acid (583-93-7), linolenic acid (463-40-1), p-aminobenzoic acid (150-13-0), methyl folate (134-35-0), prostaglandin D2 (41598-07-6), 3-methoxy tyrosine (7636-26-2), prostaglandin E1 (745-65-3), N-formyl methionine (4289-98-9), angiotensin II (11128-99-7), angiotensin III (12687-51-3), arachidonic acid (506-32-1), endorphin L (14-18-6), dihydrofolic acid (4033-27-6), isospaglumic acid (3106-85-2), [5S, 12R]-dyhydroxy-[6Z,8E, 10E, 14Z]-eicosatetraenoic acid (71160-24-2), anisic acid (100-09-4), anthranilic acid (118-92-3), N-acetyl-L-glutamine (1188-37-0), Prostaglandin F2a (551-11-1), aminomalonic acid (1068-84-4), 5-aminolevulinic acid (106-60-5), tricosanoic acid (2433-96-7), 4-trimethylammonio butyric acid (407-64-7), S-adenosyl-L-methionine (29908-03-0), leukotriene C4 (72025-60-6), dinoprostone (363-24-6), 13,14-dihydro-15-keto prostaglandin A2 (74872-89-2), succinic semialdehyde (692-29-5), thioctic acid (1200-22-2), thromboxane A2 (57576-52-0), 2-amino-3-hydroxybenzoic acid (548-93-6), niacin (59-67-6), 20-hydroxy leukotriene B4 (79516-82-8), 3-methylthiopropanoic acid (646-01-5), 3-pyridylacetic acid (501-81-5), diethylarginine (30315-93-6), 3-chlorobenzoic acid (535-80-8), 2-oxo-4-methylthiobutyric acid (583-92-6), folinic acid (58-05-9), benzoylformic acid (611-73-4), and α-hydroxyhexanoic acid (6064-63-7), and some artificially synthesized carboxyl group-containing compounds and peptides.

For example, the artificially synthesized carboxyl group-containing compounds and peptides include: diglycine derivatives. For example, the artificially synthesized carboxyl group-containing compounds and peptides include: N-Boc-diglycine, N-allyl diglycine, N-propargyl diglycine, N-benzyl diglycine, N-aryl allyl diglycine, and/or N-aryl propargyl diglycine, and ubiquitin.

For example, the active esters include: N-hydroxy succinimide activated esters, N-hydroxysulfosuccinimide activated esters, isocyanuric acid activated esters, dimethoxy-substituted isocyanuric acid activated esters, pentafluorophenol activated esters, and other active esters capable of directly reacting with amino and hydroxyl groups.

In one or more embodiments of the present disclosure, the acyl halides include: acyl chloride, acyl bromide, and acyl iodide.

In one or more embodiments of the present disclosure, the specific amino acids labeled with the ubiquitination reagent include following types: lysine, histidine, threonine, serine, tyrosine, arginine, tryptophan, 5-hydroxylysine and protein terminal amino acids.

In one or more embodiments of the present disclosure, the alkylation reagent includes aliphatic aldehydes and aromatic aldehydes; and/or alkyl halides and aryl halides.

The chemical alkylation reagent, when performing the alkylation labeling, should be capable of reacting with proteins or peptides in a mild condition, to obtain specifically modified proteins or peptides at a high yield.

In one or more embodiments of the present disclosure, the amino acids labeled with the alkylation reagent include following types: lysine, arginine, tryptophan, histidine, cysteine and protein terminal amino acids.

For example, the aliphatic aldehyde and the aromatic aldehyde include: formaldehyde, paraformaldehyde, acetaldehyde, acrolein, and benzaldehyde.

For example, the alkyl halides and the aryl halides include: iodomethane, bromomethane, chloromethane, iodoethane, bromoethane, chloroethane, allyl iodide, allyl bromide, allyl chloride, benzyl iodide, benzyl bromide, benzyl chloride, prenyl iodide, prenyl bromide, prenyl chloride, prenyl pyrophosphoric acid, prenyl methanesulfonic acid, geranyl chloride, geranyl bromide, geranyl iodide, geranyl pyrophosphoric acid, geranyl methanesulfonic acid, farnesyl iodide, farnesyl bromide, farnesyl chloride, farnesyl pyrophosphoric acid, farnesyl methanesulfonic acid, geranylgeranyl chloride, geranylgeranyl bromide, geranylgeranyl iodine, geranylgeranyl pyrophosphoric acid, and geranylgeranyl methanesulfonic acid.

In one or more embodiments of the present disclosure, the phosphorylating reagent includes one or more from a combined reagent of ATP, phosphoric acid and phosphorus pentoxide, phosphorus oxychloride, phosphorus pentachloride, diethyl allyl phosphate (3066-75-9), bis(2-cyanoethyl)-N,N-diisopropyl phosphoramidite (102690-88-0), bis[1-(2-nitrophenyl)ethyl]-N,N-diisopropyl phosphoramidite (207516-14-1), bis-trifluoromethyl ethylphosphonate (650-16-8), 3-bromethyl phosphoryl dichloride (4167-02-6), 2-(carboxyethyl)triphenyl phosphine chloride (36626-29-6), 2-chloro-1,3,2-benzodioxaphosphorin-4-one (5381-99-7), 2-cyanoethyl N,N-diisopropylchlorophosphoramidite (89992-70-1), 2-cyanoethyl dichlorophosphite (76101-30-9), bis(diisopropylamino)(2-cyanoethoxy)phosphine (102691-36-1), dibenzyl diethylphosphoramidite (67746-43-4), dibenzyl diisopropylphosphoramidite (108549-23-1), dibenzyl phosphite (538-60-3), dibenzylphosphoryl chloride (538-37-4), di-tert-butyl N,N-diethylphosphoramidite (117924-33-1), di-tert-butyl N,N-diisopropylphosphoramidite (137348-86-8), di-p-chlorophenyl N,N-diisopropylphosphoramidite (128858-43-5), diethylphosphoramidous dichloride (1069-08-5), dichloro-N,N-diisopropylphosphoramidite (921-26-6), diethyl (3-bromopropyl)phosphonate (1186-10-3), diethyl-N,N-diisopropylphosphoramidite (42053-26-9), 4-(diethylphosphono)-3-methyl-2-butenenitrile (87549-50-6), diethyl (tosyloxy)methylphosphonate (31618-90-3), dimethyl N,N-diethyl phosphoramidite (20621-25-4), dimethyl N,N-diisopropylphosphoramidite (29952-64-5), ethylphosphonic dichloride (1066-50-8), methyltriphenoxyphosphonium iodide (17579-99-6), phosphonomethanol (2617-47-2), 6-(O-phosphorylcholine)hydroxycaproic acid (73839-24-4), tetrabenzyl pyrophosphate (990-91-0), pyrophosphoric acid tris(tetrabutylammonium) potassium salt (76947-02-9), trisodium 3-O-benzyl-2-phosphonyl-D-glycerate, tetraethyl methylenediphosphonate (1660-94-2), acephate (30560-19-1), trimetaphosphate, and other compounds to which a phosphate group can be added.

When the phosphorylating labeling is carried out, the first chemical labeling can be carried out at the protein level and also can be carried out at the peptide level. A phosphorylating reagent used in the first chemical labeling should be capable of reacting with proteins in a mild condition, to obtain phosphorylated proteins at a high yield, and also can react with peptides in a mild condition, to obtain phosphorylated peptides at a high yield.

In one or more embodiments of the present disclosure, the amino acids labeled with the phosphorylating reagent include following types: threonine, serine, tyrosine, histidine and lysine.

In one or more embodiments of the present disclosure, before labeling the internal standard and the protein samples to be studied with the isobaric tandem mass tags, it further includes pre-treatment on all protein samples, wherein the pre-treatment includes precipitation, drying, and enzymolysis.

For example, methods of the precipitation include a chloroform-methanol precipitation method, a TCA precipitation method, and an acetone precipitation method.

For example, an enzyme used in the enzymolysis includes one or more from trypsin, chymotrypsin, clostripain, pepsin, rLys-C protease, Glu protease (Glu-C), endopeptidase (Lys-C), and Arg-C protease.

In one or more embodiments of the present disclosure, for the chemical modification labeling, according to different types of post-translational modifications to be detected and different isobaric tandem mass tag reagents used, the chemical labeling is carried out at the protein level, or at the peptide level.

When the isobaric tandem mass tag is labeled at a protein terminal amino group or lysine side chain, if the post-translational modifications to be studied are not on the amino group, the chemical modification can be carried out at the protein level or the peptide level; if the post-translational modifications to be detected are not on the amino group, the chemical labeling is carried out at the protein level.

When the isobaric tandem mass tag is labeled on the thiol group of cysteine, and the post-translational modifications to be studied are not on the thiol group of cysteine, the chemical labeling can be carried out at the protein level or the peptide level.

Generally, when the chemical labeling is carried out at the protein level, in order to achieve higher labeling efficiency, the protein needs to be denatured, by reduction to break a disulfide linkage of the protein, and then alkylating the thiol groups, preventing free thiol groups from re-generating a disulfide bond again. Only when the proteins are completely reduced and denatured, the labeling efficiency of each sample can be ensured to be completely consistent, and if the reduction is not thorough, the labeling efficiency may be inconsistent due to influence of secondary structure, which inevitably also will affect the result of final characterization and quantitation. In certain special situations, in order to reduce interference of additives from outside, the reduction and alkylation may be not carried out.

Therefore, in one or more embodiments of the present disclosure, before labeling with the isobaric tandem mass tags, the protein extracts for making internal standard and the protein samples to be detected are subjected to reduction, and/or alkylation.

For example, when the isobaric tandem mass tag is labeled on the protein terminal amino group or lysine, a reduction reagent used in the reduction includes DTT and TCEP, and the alkylating reagent used in the alkylation includes iodoacetamide.

In one or more embodiments of the present disclosure, when the isobaric tandem mass tag is labeled on the cysteine thiol group, a reduction reagent includes DTT and TCEP, and the alkylating reagent is an isobaric labeling reagent.

When the post-translational modification is studied, in a process of performing the first chemical labeling of the internal standard, the chemical labeling reagent is excessive, while residue of the excessive chemical labeling reagent will affect the labeling efficiency of the isobaric tandem mass tag labeling reagent, thus affecting preciseness of the quantitation result. Therefore, in one or more embodiments of the present disclosure, after the chemical labeling, excessive labeling reagent is quenched using a quenching reagent.

In one or more embodiments of the present disclosure, when the chemical labeling reagent is an acylating reagent, the quenching reagent is one or more selected from hydroxylamine, ammonia water, Tris base, and primary amine and secondary amine compounds, for example, hydroxylamine and/or aqueous ammonia.

For example, when the chemical labeling reagent is an alkylating reagent, for example, NaBH₃CN, the quenching reagent is formic acid.

For example, when the chemical labeling reagent is a phosphorylating reagent or an adenylating reagent, the quenching reagent is a basic compound, for example, one or more selected from triethylamine, NaOH and NaHCO₃.

In one or more embodiments of the present disclosure, after labeling with the isobaric tandem mass tags, the excessive reagent also need to be quenched. Moreover, after labeling all samples and quenching, all samples were combined and desalted to reduce influences of salt and some other small-molecule impurities in the samples to HPLC separation and mass spectrometry detection. For example, in one or more embodiments of the present disclosure, the combined samples are desalted using a C18 desalting column, so as to reduce influences of salt and some other small-molecule impurities in the samples to the mass spectrometry detection.

In one or more embodiments of the present disclosure, in order to obtain a detection result with a higher throughput, in the present disclosure, after the samples are combined and desalted, peptides are separated into fractions using HPLC according to different hydrophilicity and hydrophobicity of the peptides.

In one or more embodiments of the present disclosure, following the tandem mass spectrometry analysis, mass spectrometry data is further processed using proteomic software, for example, MaxQuant, PD and Mascot, to obtain qualitative and quantitative data of the proteins and post-translational modifications.

The detection and quantitation method for proteomics of post-translational modification as mentioned in the above is used in qualitative and quantitative analysis of protein post-translational modifications of trace biological samples.

The present disclosure further provides a method for diagnosing or detecting diseases related to the dysregulation of post-translational modifications, including performing detection and quantitation for proteomics of post-translational modifications in subject samples.

In one or more embodiments, the proteomics of post-translational modifications includes at least one from the following: acylation, alkylation, phosphorylation, ubiquitylation, glycosylation, sulfation, selenylation, S-nitrosylation, adenylation, hydroxylation, citrullination, carbamylation, amidation and iodization.

In one or more embodiments, the diseases related to the dysregulation of post-translational modifications include at least one from cancers, immunological diseases, cardiovascular diseases, neurodegenerative lesions, muscular dystrophy, infectious diseases and metabolic syndromes.

In one or more embodiments, the diseases related to the dysregulation of post-translational modifications are cancers.

Embodiments of the present disclosure will be described in detail below in combination with examples, while a person skilled in the art will understand that the following examples are merely for illustrating the present disclosure, but should not be considered as limiting the scope of the present disclosure. If no specific conditions are specified in the examples, they are carried out under normal conditions or conditions recommended by the manufacturer. If the manufacturers of reagents or apparatus used are not specified, they are conventional products commercially available.

### Example 1 Comparative Acetylomics and 2-Hydroxyisobutyrylomics in Livers of Mice on a High Fat Diet

(1) Mice feeding. The mice were divided into two groups in equal number, wherein one group was fed normally, and the other group was fed on a high fat diet, both being fed for 16 weeks.

(2) Collection of mouse liver tissues. The experiment mice were anesthetized at a dosage of 0.04 mL/10 g (an anaesthetic was 10% chloral hydrate), and the mouse livers were perfused with a 1×PBS solution (8.0 g/L sodium chloride, 0.2 g/L potassium chloride, 2.72 g/L disodium hydrogen phosphate heptahydrate, and 0.245 g/L potassium dihydrogen phosphate) through hepatic portal veins, followed by harvesting liver tissues, which were quickly frozen in liquid nitrogen, and stored at −80° C. for subsequent use.

(3) Protein Extraction from the Mouse Liver Tissues.

1 mL of a PBS lysing solution (a PBS solution containing 1% NP-40, 0.5% sodium deoxycholate, 25 mM nicotinamide, 10 mM sodium butyrate, 1× protease inhibitor (cocktail), 1× phosphatase inhibitor A solution, and 1× phosphatase inhibitor B solution) was added to 100 mg of the mouse liver tissue sample, and the tissues were smashed with a tissue homogenizer. The smashed tissues were subjected to supersonic dissociation, and high-speed low-temperature centrifugation (20000 g, 30 min, 4° C.). A supernatant was taken, and a protein concentration was measured using a BrandFord method.

(4) Experimental Samples.

The internal standard: 50 μg of protein extracts from the liver tissues of the normally fed mice and the liver tissues of the mice on a high fat diet mixed at 1:1 were used for each modification;

The protein samples to be studied: 50 μg of protein extracts from the liver tissues of the normally fed mice with duplicates, 50 μg of protein extracts from the liver tissues of the mice on a high fat diet with duplicates.

(5) Reduction and alkylation of proteins. 100 mM TEAB solution was added to 50 μg of protein extracts from each sample to adjust the volume to be 50 μL, and 2.5 μL of 200 mM TCEP was added respectively for vertex blending, followed by incubation at 55° C. for 1 h. When the samples were cooled to a room temperature, 2.5 μl of 375 mM iodoacetamide solution (freshly made) was added respectively to the above samples, and the mixtures were shaken in the dark for 30 min at a room temperature.

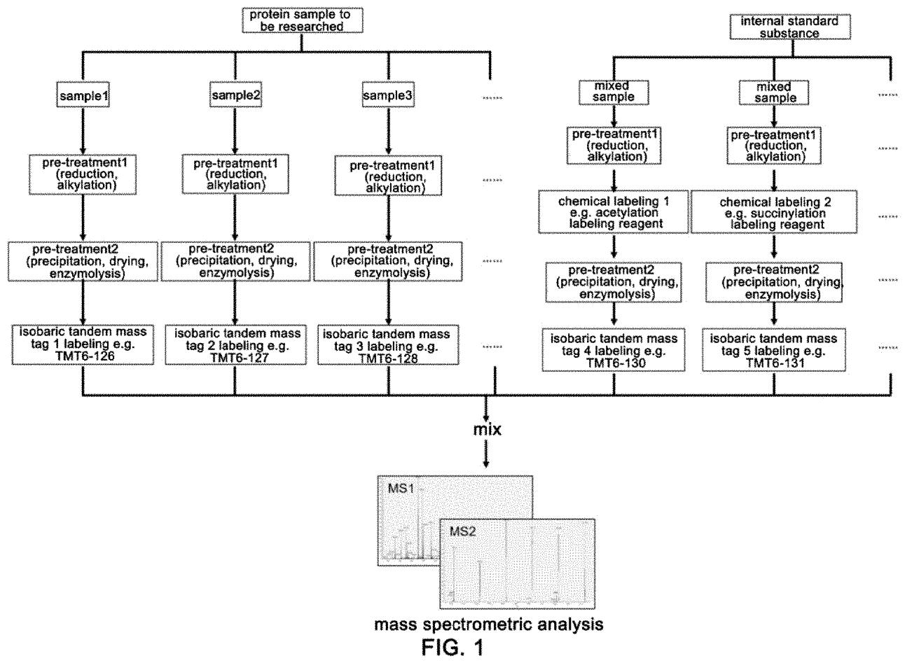

(6) 1 mg of Hib-NHS (N-hydroxysuccinimide activated 2-hydroxyisobutyric acid) and 1 mg of Ac-NHS (N-hydroxysuccinimide activated acetic acid) were respectively dissolved in 2 μL of DMSO, and respectively added to chemically label the internal standard samples, followed by incubation at a room temperature for 2 h (FIG. 1).

(7) After the labeling was completed, 2.1 μL of 50% hydroxylamine was added to the groups of internal standard substance, for incubation at a room temperature for 2 h. This step may be omitted in a specific situation.

(8) Protein sample precipitation. To the six groups of protein samples, i.e. the two groups of internal standard substance and the four groups of protein samples to be researched, methanol of 4 times volume, chloroform of 1 time volume and water of 3 times volume were added respectively, and the mixtures were subjected to vortex centrifugation (10000 g, 10 min). After the centrifugation, the liquid was divided into three layers, i.e. an aqueous phase layer, a protein layer, and a chloroform layer in sequence. An upper-layer mixed phase of methanol and water was gently removed, and methanol of 4 times volume was added to the six groups of samples respectively, and the mixtures were subjected to vortex centrifugation (20000 g, 10 min). A supernatant was gently removed, and an organic reagent remaining in the protein samples was removed by volatilization at a room temperature.

(9) Tryptical digestion. All six samples were respectively dissolved in 50 μL of 50 mM TEAB, and 1 μg of trypsin was added thereto respectively. The mixtures were subjected to tryptical digestion at 37° C. overnight.

(10) After the tryptical digestion was completed, the two internal standard samples were respectively desalted using 10 mg of C18 column. This step may be omitted in a specific situation.

(11) 50 μL of 50 mM TEAB solution was added again to the internal standard samples, respectively, to dissolve the samples.

(12) TMT labeling.

The six samples were subjected to labeling with TMT⁶ reagent, respectively. Specific dosages of the TMT reagent are as follows (Table 1)

TABLE 1
Distribution and Dosage of TMT⁶ Reagent for
Comparative Acetylomics and 2-Hydroxyisobutyrylomics
in Livers of Mice on High Fat Diet

| | | | TMT |
|---|---|---|---|
| | | Sample | Reagent |
| TMT Reagent | Sample | Amount | Dosage |
| TMT⁶-126 | internal standard substance (Kac) | 50 μg | 0.4 mg |
| TMT⁶-127 | internal standard substance (Khib) | 50 μg | 0.4 mg |
| TMT⁶-128 | liver tissues-1 of normally fed mice | 50 μg | 0.4 mg |
| TMT⁶-129 | liver tissues-1 of mice on high fat | 50 μg | 0.4 mg |
| | diet | | |
| TMT⁶-130 | liver tissues-2 of normally fed mice | 50 μg | 0.4 mg |
| TMT⁶-131 | liver tissues-2 of mice on high fat | 50 μg | 0.4 mg |
| | diet | | |

(13) The six samples were quenched, mixed, concentrated, and desalted using a C18 desalting column.

(14) The mixed samples were separated into 20 fractions by preparative chromatography using C18 column.

Chromatography liquid phase: A phase: 98% H₂O, 2% acetonitrile, 10 mM ammonium formate, pH=10:

B phase: 10% H₂O, 900 acetonitrile, 10 mM ammonium formate, pH=10.

300 μL of A phase solution was added to the sample to dissolve the sample, followed by centrifugation (10000 g, 20 min). A supernatant was taken and injected. Chromatography conditions are as follows (Table 2):

TABLE 2
Samples-Grouping Chromatography Conditions for
Comparative Acetylomics and 2-hydroxyisobutyrylomics
in Livers of Mice on High Fat Diet

| Time | A phase (%) | B phase (%) | Flow rate (mL/min) |
|---|---|---|---|
| 0 | 100 | 0 | 1.000 |
| 10 | 95 | 5 | 1.000 |
| 80 | 65 | 35 | 1.000 |
| 95 | 40 | 60 | 1.000 |
| 105 | 30 | 70 | 1.000 |
| 120 | 0 | 100 | 1.000 |

One tube of sample was collected every minute, starting from a first tube of eluate collected, every 19 tubes were combined into one fraction, 20 fractions in total. They were concentrated and dried respectively.

(15) The 20 fractions were desalted with a Zip-tip C18 column. Each sample needed to be subjected to two times of desalting operation, and eluates after the two times of desalting were combined, and concentrated and dried.

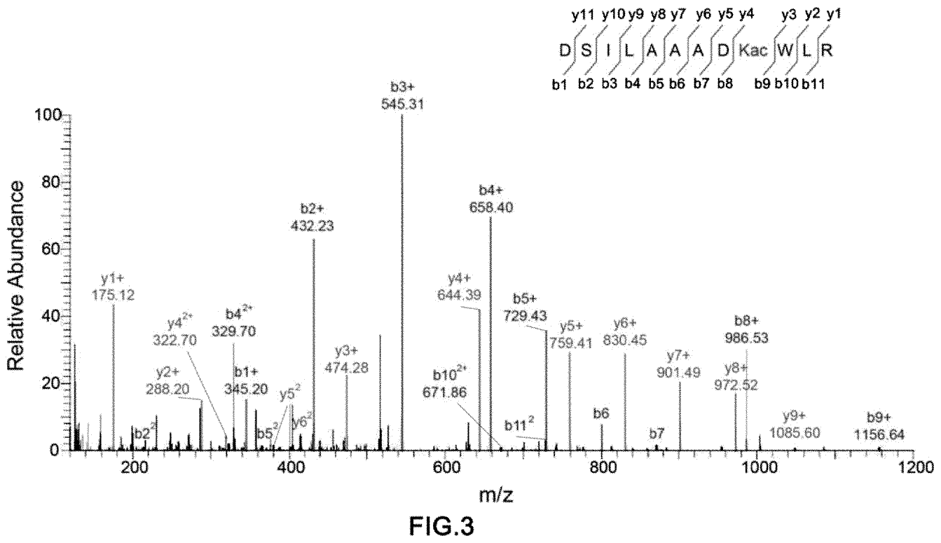

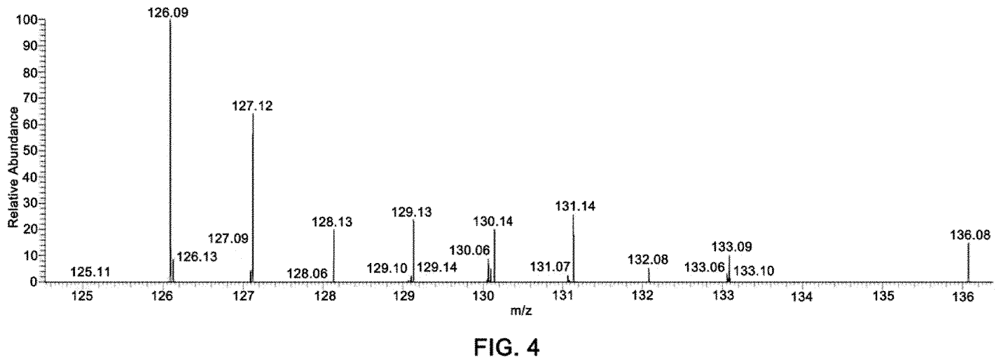

(16) The 20 fractions were subjected to LC-MS/MS analysis. Mass spectrometric data was further processed and analyzed using software such as MaxQuant, PD and Mascot. An example of MS/MS spectrum of acetylated peptide was shown in FIG. 3, and the peptides were quantified by reporter ions in the low mass range (FIG. 4).

4815 proteins in total were identified with this method, wherein 4679 proteins could be quantified, and 122 proteins had significant differences in expression (an average protein abundance ratio was greater than 1.33 or less than 0.75). The number of proteins and peptides to which acetylation and 2-hydroxyisobutyrylation occurred are as shown in Table 3.

TABLE 3
Number of Modified Proteins and Peptides for Comparative Acetylomics
and 2-hydroxyisobutyrylomics in Livers of Mice on High Fat Diet

| | | | | | | Number of |
|---|---|---|---|---|---|---|
| | | | | Protein Types | | Peptides |
| | | Number of | Number of | Corresponding | Number of | with |
| Modification | Protein | Modification | Identified | to Identified | Quantifiable | Significant |
| Type | Type | Sites | Peptides | Peptides | Peptides | Differences |
| Kac | 1253 | 1 | 1914 | 1005 | 1888 | 308 |
| | | 2 | 1000 | 682 | 993 | 130 |
| Khib | 1254 | 1 | 1927 | 1064 | 1794 | 102 |
| | | 2 | 930 | 650 | 598 | 52 |

Notes: the number of peptides with significant differences was the number of peptides with an intensity ratio (high fat diet fed/normally fed) less than 0.75 or greater than 1.33.

### Example 2 Comparative Dimethylomics in Livers of Mice on a High Fat Diet

(1) Mice feeding. (The same as Example 1)

(2) Collection of mouse liver tissues. (The same as Example 1)

(3) Protein extraction from the mouse liver tissues. (The same as Example 1)

(4) Experimental Samples.

The internal standard: 50 μg of protein extracts from the liver tissues of the normally fed mice and the liver tissues of the mice on a high fat diet mixed at 1:1.

The protein samples to be researched: 50 gig of protein extracts from the liver tissues of the normally fed mice with duplicates, 50 μg of protein extracts from the liver tissues of the mice on a high fat diet with duplicates.

(5) Reduction and alkylation of protein samples. (The same as Example 1)

(6) Methylation labeling of the group of internal standard substance.

8 μL of 4% (v/v) CH₂O aqueous solution was added to the internal standard sample, and the mixture was subjected to vortex blending; then 8 μL of 0.6 M NaBH₃CN was added, and the mixture was subjected to vortex blending, and reaction at a room temperature for 1 h, subsequently, 32 μL of quencher (1% (v/v) aqueous ammonia) was added, and the mixture reacted at a room temperature for 2 h.

(7) Protein sample precipitation. (The same as Example 1)

(8) Tryptical digestion. (The same as Example 1)

(9) After the tryptical digestion was completed, the internal standard sample was desalted using 10 mg of C18 desalting column. This step may be omitted in a specific situation.

(10) TMT labeling. The five samples, i.e. the internal standard and the samples to be studied, were labeled with TMT⁶ tags, respectively. Specific dosages of the TMT reagent are as follows (Table 4).

TABLE 4
Distribution and Dosage of TMT⁶ Reagent for Comparative
Dimethylomics in Livers of Mice on High Fat Diet

| | | | TMT |
|---|---|---|---|
| | | Sample | Reagent |
| TMT Reagent | Sample | Amount | Dosage |
| TMT⁶-126 | Internal standard substance (K | 50 μg | 0.4 mg |
| | dimethylation) | | |
| TMT⁶-127 | Blank | 0 | 0 |
| TMT⁶-128 | liver tissues-1 of normally fed mice | 50 μg | 0.4 mg |
| TMT⁶-129 | liver tissues-11 of mice on high fat | 50 μg | 0.4 mg |
| | diet | | |
| TMT⁶-130 | liver tissues-2 of normally fed mice | 50 μg | 0.4 mg |
| TMT⁶-131 | liver tissues-12 of mice on high fat | 50 μg | 0.4 mg |
| | diet | | |

(11) The five samples were quenched, mixed, concentrated, and desalted using a C18 desalting column.

(12) The desalted samples were separated and divided into 20 fractions using high performance liquid chromatography, and respectively desalted with a Zip-tip C18 column. (The same as Example 1)

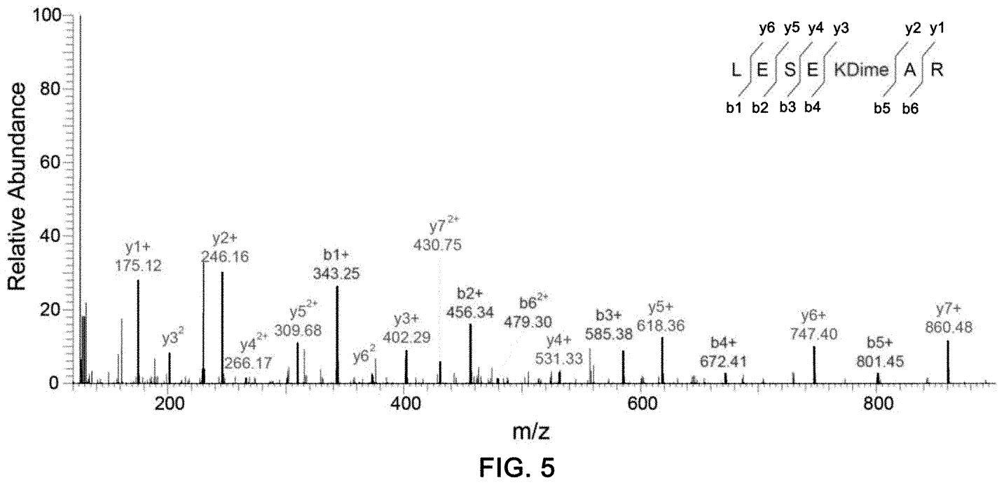

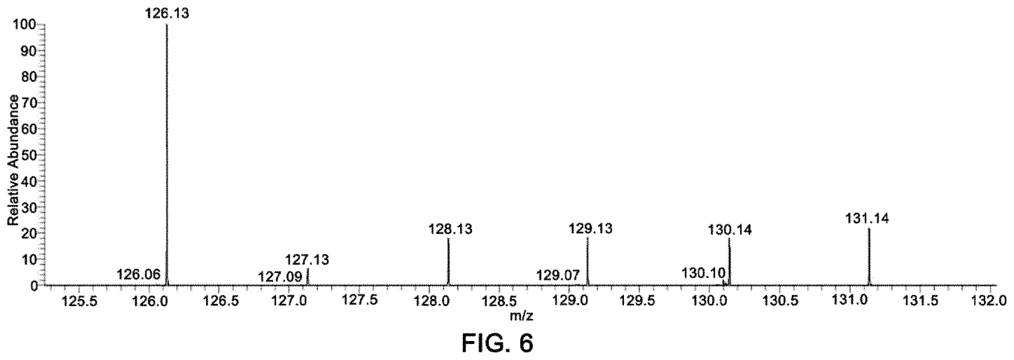

(13) LC-MS/MS analysis was performed, wherein mass spectrometric data was further processed and analyzed using software such as MaxQuant, PD and Mascot. An example of MS/MS spectrum of dimethylated peptide was shown in FIG. 5, and the peptides were quantified by reporter ions in the low mass range (FIG. 6).

5244 proteins in total were identified through this method, wherein 5080 proteins could be quantified, and 335 proteins had significant differences in expression (an average protein abundance ratio was greater than 1.33 or less than 0.75). Lysine dimethylation was found on 1451 proteins, and 1314 peptides (771 proteins) had one dimethylation site, wherein 1239 peptides could be quantified. 133 peptides belonging to 118 proteins had two dimethylation sites, wherein 121 peptides could be quantified. Besides, the dimethylation levels of 187 peptides had significant differences between high-diet fed mice and normally fed mice (a peptide abundance ratio was greater than 1.33 or less than 0.75).

### Example 3 Quantitative Phosphoproteomic Analysis of Colorectal Cancer Sample

(1) Obtaining colorectal cancer clinical samples: all colorectal cancer clinical samples came from patients with colorectal cancer having received surgical excision and treatment in Department of Oncology of West China Hospital of Sichuan University;

(2) Protein extraction: 100 mg of 3 pairs of colorectal cancer tissues (tumorous tissues and paired non-tumorous tissues) were taken, 1 mL of RIPA lysing buffer (1% NP-40, 0.5% sodium deoxycholate, 150 mM sodium chloride, 50 mM Tris (pH=7.5), 25 mM nicotinamide, 10 mM sodium butyrate, 1× protease inhibitor (cocktail), 1× phosphatase inhibitor A solution, and 1× phosphatase inhibitor B solution) was added, and the tissues were smashed with a tissue homogenizer. The smashed tissues were subjected to supersonic dissociation, and high-speed centrifugation (20000 g, 30 min, 4° C.). The supernatant of each sample was collected, and the protein concentration of each sample was measured using a BrandFord method.

(3) Experimental Samples.

The protein sample to be studied: three pairs (tumorous tissues and paired adjacent non-tumorous tissues) of colorectal cancer clinical samples, with 50 μg of proteins for sample.

The internal standard: 50 μg of protein extracts from three pairs of tissues mixed at 1:1:1:1:1:1.

(4) Reduction and alkylation of proteins. (The same as Example 1)

(5) Protein precipitation. (The same as Example 1)

(6) Tryptical digestion. (The same as Example 1)

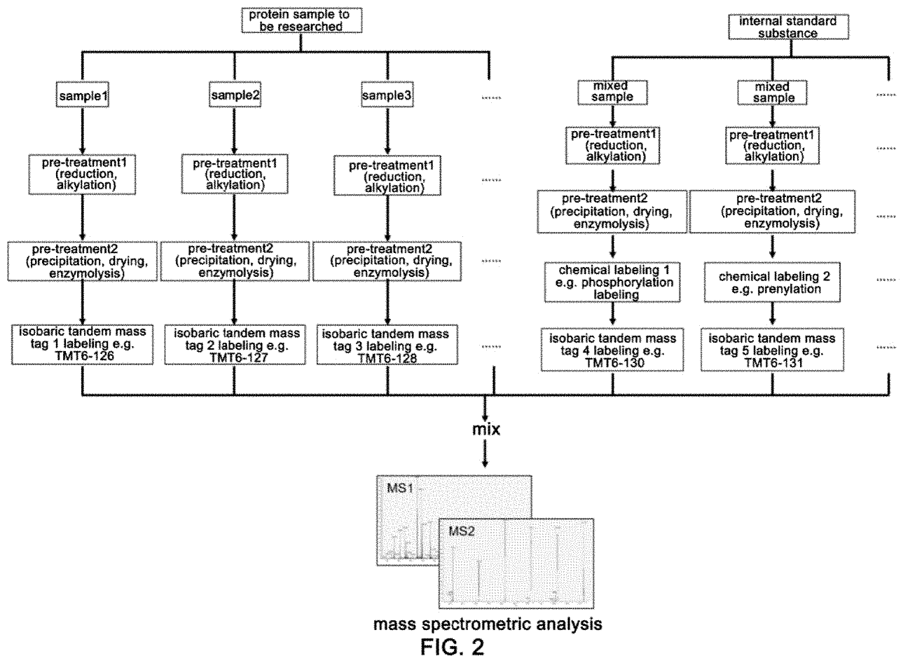

(7) The internal standard was subjected to phosphorylation labeling. The peptides having undergone tryptical digestion were concentrated and dried. 50 μL of dry tetrahydrofuran, 1 μL of triethylamine, and 1 μL phosphorus oxychloride were added and incubated at 4° C. for 4 h, And then, 20 μL of water was added, followed by incubation at 4° C. for 8 h. After concentration and drying, 50 mM TEAB buffer was added for TMT labeling (FIG. 2).

(8) TMT labeling. All the seven samples, i.e. the internal standard and the sample to be studied, were subjected to TMT reagent labeling. Specific dosages of the TMT reagent are as follows (Table 5).

TABLE 5
Distribution and Dosage of TMT¹⁰ Reagent for Quantitative
Phosphorylomics of Three Groups of Colorectal Cancer Sample

| | | | TMT |
|---|---|---|---|
| | | Sample | Reagent |
| TMT Reagent | Sample | Amount | Dosage |
| TMT¹⁰-126 | colon cancer tissues-1 | 50 μg | 0.4 mg |
| TMT¹⁰-127C | internal standard (phosphorylation) | 50 μg | 0.4 mg |
| TMT¹⁰-127N | colon cancer tissues-2 | 50 μg | 0.4 mg |
| TMT¹⁰-128C | colon cancer tissues-3 | 50 μg | 0.4 mg |
| TMT¹⁰-128N | blank | 0 | 0 |
| TMT¹⁰-129C | Paired non-tumor sample-1 | 50 μg | 0.4 mg |
| TMT¹⁰-129N | blank | 0 | 0 |
| TMT¹⁰-130C | Paired non-tumor sample-2 | 50 μg | 0.4 mg |
| TMT¹⁰-130N | blank | 0 | 0 |
| TMT¹⁰-131 | Paired non-tumor sample-3 | 50 μg | 0.4 mg |

(9) The labeled seven samples were quenched, mixed, concentrated, and desalted with a C18 desalting column.

(10) The desalted samples were separated using high performance liquid chromatography and combined into 20 fractions, and desalted with a Zip-tip C18 column. (The same as Example 1)

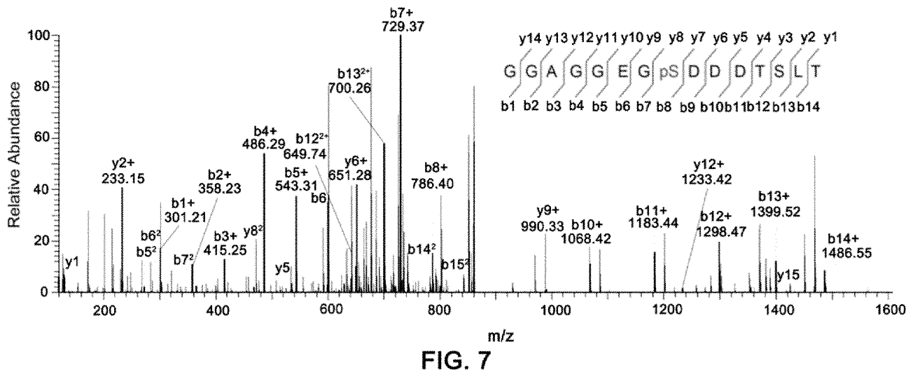

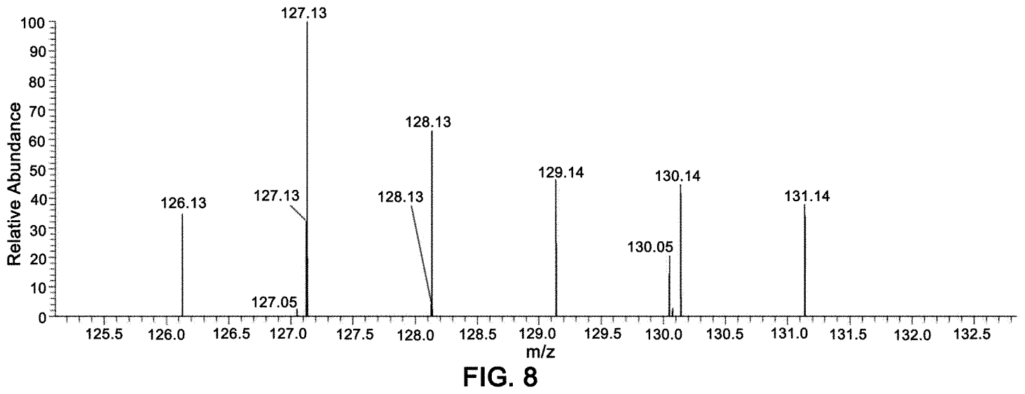

(11) LC-MS/MS analysis, wherein mass spectrometry data was further processed and analyzed using software such as MaxQuant, PD and Mascot. An example of MS/MS spectrum of phosphorylated peptide was shown in FIG. 7, and the peptides were quantified by reporter ions in the low mass range (FIG. 8).

2965 phosphorylated peptides in total were identified with this method, wherein there were 2820 peptides each of which had one phosphorylation site, including serine phosphorylation site in 1878 peptides, threonine phosphorylation site in 904 peptides and tyrosine phosphorylation site in 38 peptides, there were 139 peptides each of which had 2 phosphorylation sites, including serine phosphorylation site in 118 peptides, threonine phosphorylation site in 77 peptides, and tyrosine phosphorylation site in 83 peptides, and there were 6 peptides each of which had 3 serine phosphorylation sites.

### Example 4 Quantitative Acetylomic Analysis of Colorectal Cancer Samples

(1) Obtaining colorectal cancer clinical samples. (The same as Example 3)

(2) Protein extraction. (The same as Example 3)

(3) Experiment Samples.

The protein sample to be researched: one pair of tumor tissue and paired carcinoma tissue, with 50 μg of protein extracts from each sample.

The internal standard: 50 μg of protein extracts from tumor and non-tumorous tissue mixed at 1:1.

(4) Reduction and alkylation of proteins. (The same as Example 1)

(5) 20 μg of Ac-NHS (N-hydroxysuccinimide activated acetic acid) were dissolved in 2 μL of DMSO, and added to the proteins used as internal standard for chemical labeling, followed by incubation at a room temperature for 2 h.

(6) Protein sample precipitation. (The same as Example 1)

(7) Enzymolysis. (The same as Example 1)

(8) TMT labeling. The internal standard sample, and the two to-be-studied samples were subjected to TMT reagent labeling. Specific dosages of the TMT reagent are as follows (Table 6).

TABLE 6
Distribution and Dosage of TMT Reagent for Quantitative
Acetylomic Analysis of Colorectal Cancer Samples

| | | | TMT |
|---|---|---|---|
| | | Sample | Reagent |
| TMT Reagent | Sample | Amount | Dosage |
| TMT⁶-126 | Internal standard sample | 50 μg | 0.4 mg |
| TMT⁶-127 | Blank | 0 | 0 |
| TMT⁶-128 | Blank | 0 | 0 |
| TMT⁶-129 | Tumor sample | 50 μg | 0.4 mg |
| TMT⁶-130 | Blank | 0 | 0 |
| TMT⁶-131 | Paired non-tumor sample | 50 μg | 0.4 mg |

(9) The isobarically labeled samples were quenched, mixed, concentrated, and desalted with a C18 desalting column.

(10) The desalted samples were separated and divided into 20 groups using high performance liquid chromatography, and respectively desalted with a Zip-tip C18 column. (The same as Example 1)

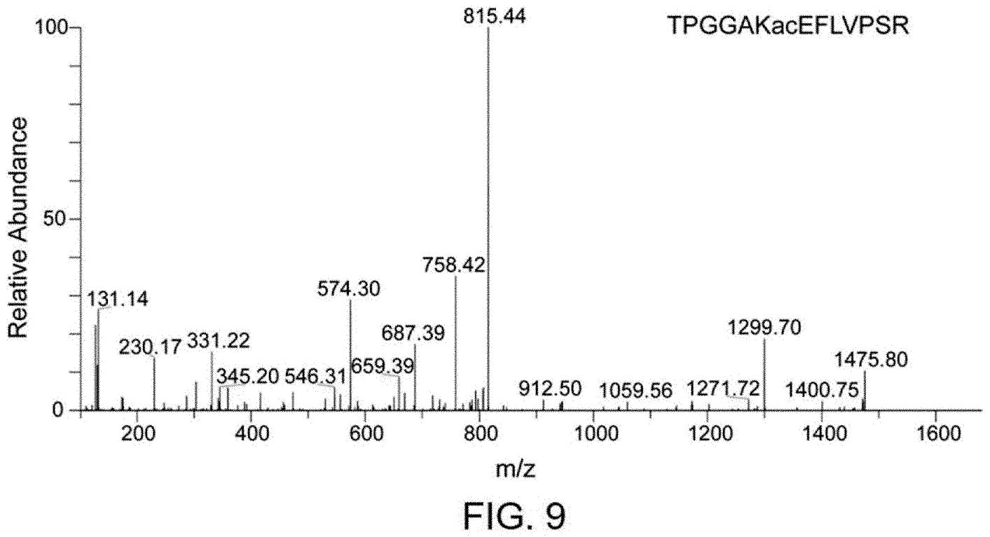

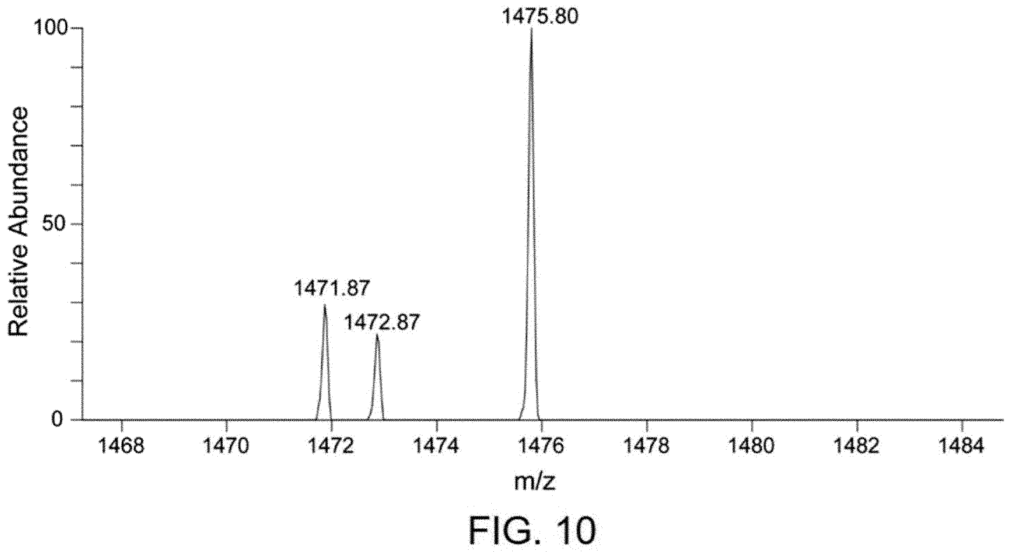

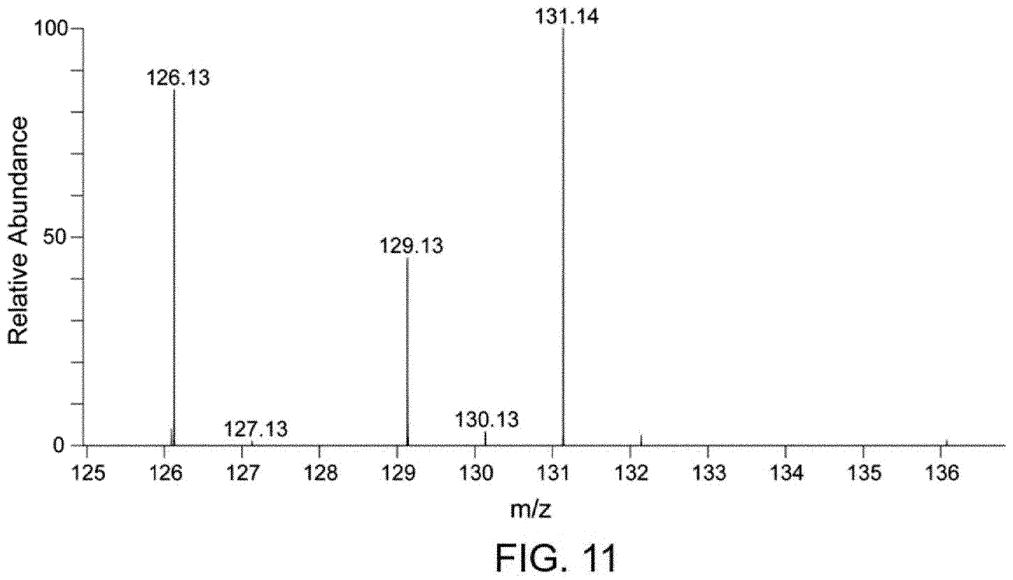

(11) LC-MS/MS analysis, wherein mass spectrometry data was further processed and analyzed using software such as MaxQuant, PD and Mascot An example of MS/MS spectrum of acetylated peptide was shown in FIG. 9 The characterization of acetylation was determined by the TMTc reporters (FIG. 10), and relative acetylated peptide levels between the two colorectal cancer tissue samples were measured by low mass range reporter ions (129 and 131)(FIG. 11).

As a result of data processing and analysis, 2770 acetylated peptide in total were quantified with this method.

Finally, it should be indicated that the various examples above are merely for illustrating the technical solutions of the present disclosure, rather than limiting the present disclosure; while the detailed description is made to the present disclosure with reference to various preceding examples, those ordinarily skilled in the art should understand that they still could modify the technical solutions recited in the various preceding examples, or make equivalent substitutions to some or all of the technical features therein; these modifications or substitutions do not make the corresponding technical solutions essentially depart from the scope of the technical solutions of the various examples of the present disclosure.

1. A detection and quantitation method for proteomics of post-translational modifications, comprising:
labeling protein samples to be detected and an internal standard with isobaric tandem mass tags at peptide level, and
carrying out tandem mass spectrometry analysis of a peptide mixture of the labeled protein samples to be detected and the labeled internal standard,
wherein the internal standard is a peptide mixture rich in one or more post-translational modifications to be detected.

2. The detection and quantitation method for proteomics of post-translational modifications according to claim 1, wherein the method comprises performing enzymolysis on the protein samples to be detected in advance to generate peptides, labeling the peptides and the internal standard respectively with the isobaric tandem mass tags, mixing the labeled peptides and the labeled internal standard, performing tandem mass spectrometry analysis, and performing qualitative and quantitative analysis on the one or more post-translational modifications using reporter ions,
wherein the reporter ions include reporter ions in low mass range and complementary reporter ions containing peptide sequences in high mass range.

3. The detection and quantitation method for proteomics of post-translational modifications according to claim 1, wherein types of the one or more post-translational modifications to be detected comprise at least one of: acylation, alkylation, phosphorylation, ubiquitination, glycosylation, sulfation, selenylation, S-nitrosylation, adenylation, hydroxylation, iodization, citrullination, carbamylation and amidation.

4. The detection and quantitation method for proteomics of post-translational modifications according to claim 1, wherein the isobaric tandem mass tags comprise at least one of TMT, iTRAQ, DiART, CIT, CILAT, DiLeu, IPTL, QITL, IVTAL, and EASI-TAG.

5. The detection and quantitation method for proteomics of post-translational modifications according to claim 1, wherein the peptide mixture is a peptide mixture obtained by chemical modification labeling of protein extracts, and then enzymolysis of the modified proteins, and/or
a peptide mixture obtained by enzymolysis of protein extracts, and then chemical modification labeling of the digested proteins, and/or
an artificially synthesized peptide mixture containing the one or more post-translational modifications to be detected, and/or
a peptide mixture obtained by enriching post-translational modifications to be detected from a peptide mixture using antibodies or other enrichment reagents.

6. The detection and quantitation method for proteomics of post-translational modifications according to claim 5, wherein an enzyme used in the enzymolysis comprises one or more selected from the group consisting of trypsin, chymotrypsin, clostripain, pepsin, rLys-C protease, Glu protease (Glu-C), endopeptidase (Lys-C) and Arg-C protease.

7. The detection and quantitation method for proteomics of post-translational modifications according to claim 5, wherein the chemical modification labeling comprises performing chemical modification, by using a chemical labeling reagent, on the protein extracts or the peptides obtained by enzymolysis of the protein extracts.

8. The detection and quantitation method for proteomics of post-translational modifications according to claim 7, wherein the chemical modification labeling reagent comprises at least one selected from the group consisting of: acylating reagent, alkylating reagent, phosphorylating reagent, glycosylating reagent, ubiquitination reagent, sulfation reagent, selenylation reagent, adenylation reagent, S-nitrosylation reagent, hydroxylation reagent, carbamylation reagent, iodization reagent, amidation reagent and enzymes causing the modification labeling.

9. The detection and quantitation method for proteomics of post-translational modifications according to claim 5, wherein the protein extracts are from one or more protein samples to be detected, and/or from one or more protein samples containing more protein species than that in the samples to be detected, and/or a mixture of recombinant proteins.

10. The detection and quantitation method for proteomics of post-translational modifications according to claim 7, wherein after the chemical modification labeling, excessive modification labeling reagent is quenched using a quenching reagent.

11. The detection and quantitation method for proteomics of post-translational modifications according to claim 8, wherein the acylating reagent comprises: derivatives of fatty acids and/or aromatic acids;
the derivatives comprising one or more selected from the group consisting of active esters, acyl halides, anhydrides, acyl coenzyme A, and high-energy compounds capable of reacting with primary amino group, secondary amino group and hydroxyl group of an amino acid.

12. The detection and quantitation method for proteomics of post-translational modifications according to claim 8, wherein the alkylating reagent comprises at least one selected from aliphatic aldehydes and aromatic aldehydes; and/or alkyl halides and aryl halides.

13. The detection and quantitation method for proteomics of post-translational modifications according to claim 8, wherein the phosphorylating reagent comprises at least one selected from the group consisting of ATP, phosphoric acid and phosphorus pentoxide, phosphorus oxychloride, phosphorus pentachloride and trimetaphosphate.

14. The detection and quantitation method for proteomics of post-translational modifications according to claim 7, wherein
when the isobaric tandem mass tags are labeled at a protein terminal amino group or lysine side chain, the chemical modification is able to be carried out at an protein level or a peptide level if the modifications to be detected are not on the amino groups; or the chemical labeling is carried out at the protein level if the modifications to be detected are on the amino groups;
when the isobaric tandem mass tags are labeled on a thiol group of cysteine and the modifications to be detected are not on the thiol group of cysteine, the chemical modification labeling is able to be carried out at the protein level or the peptide level.

15. The detection and quantitation method for proteomics of post-translational modifications according to claim 14, wherein before labeling with the isobaric tandem mass tags, the protein samples to be studied and the protein samples used for making internal standard are subjected to reduction, and/or alkylation.

16. The detection and quantitation method for proteomics of post-translational modifications according to claim 1, before labeling the protein samples to be detected and protein samples used for making internal standard with the isobaric tandem mass tags, pre-treatment on the these protein samples, wherein the pre-treatment comprises precipitation, drying and/or enzymolysis.

17. A method for diagnosing or detecting a disease related to the dysregulation of post-translational modifications, comprising performing detection and quantitation on post-translational modifications in the subject samples using the method of claim 1.

18. The method according to claim 17, wherein the post-translational modification comprises at least one selected from the group consisting of: acylation, alkylation, phosphorylation, ubiquitylation, glycosylation, sulfation, selenylation, S-nitrosylation, adenylation, hydroxylation, citrullination, carbamylation, iodization and amidation.

19. The method according to claim 17, wherein the disease comprises at least one selected from the group consisting of cancers, immunological diseases, cardiovascular diseases, neurodegenerative lesions, muscular dystrophy, infectious diseases and metabolic syndromes.
